(19) United States
(12) Patent Application Publication
(10) Pub. No.: US 2025/0149140 A1
(43) Pub. Date: May 8, 2025

(54) MEDICAL EXAMINATION SUPPORT APPARATUS, MEDICAL EXAMINATION SUPPORT METHOD, AND NON-TRANSITORY COMPUTER-READABLE MEDIUM

(71) Applicant: CANON MEDICAL SYSTEMS CORPORATION, Tochigi (JP)

(72) Inventors: Atsuko SUGIYAMA, Nasushiobara (JP); Kazuki UTSUNOMIYA, Nasushiobara (JP)

(73) Assignee: CANON MEDICAL SYSTEMS CORPORATION, Tochigi (JP)

(21) Appl. No.: 18/941,955

(22) Filed: Nov. 8, 2024

(30) Foreign Application Priority Data
Nov. 7, 2023 (JP) 2023-190149
Oct. 31, 2024 (JP) 2024-191503

(51) Int. Cl. G16H 15/00; G16H 10/60

(52) U.S. Cl. CPC G16H 15/00; G16H 10/60

(57) ABSTRACT

A medical examination support apparatus according to an embodiment includes processing circuitry. The processing circuitry obtains text information representing different kinds of medical records at different time points, the medical records associated with examination information of a patient. The processing circuitry sets imperative-sentence information representing an imperative sentence to be input to a large language model, based on the text information. The processing circuitry determines tag candidate information representing a tag candidate to be added to the text information, based on the imperative-sentence information. The processing circuitry outputs the tag candidate information.

### CROSS-REFERENCE TO RELATED APPLICATION

This application is based upon and claims the benefit of priority from Japanese Patent Application No. 2023-190149, filed on Nov. 7, 2023; and Japanese Patent Application No. 2024-191503, filed on Oct. 31, 2024; the entire contents of which are incorporated herein by reference.

### FIELD

Embodiments described herein relate generally to a medical examination support apparatus, a medical examination support method, and a non-transitory computer-readable medium.

### BACKGROUND

Conventionally, the use of a machine learning model for text data processing is known. In some applications, special and domain-specific wording or language may be contained in text data to be analyzed. For example, medical science adopts a large number of technical terms, and same wording is often used in a different manner in the medical science and in a more general field. To annotate text data, for example, tags are added to the sentences and paragraphs of the text according to predefined classification items.

In annotation work, annotated data or a knowledge base containing an accumulation of user-input medical terms can be utilized to present tag candidates for selection, for example. For such annotation task, tagging is feasible to some extent by rule-based or machine-learning natural language processing (NLP) technique. For another example, imperative sentences for labelling and text processing are easily understandably described for a large language model to make a task request for implementing some degree of tagging.

However, such techniques tend to basically add the same tags to the same sentences or words so that it may be difficult to perform tagging with each patient's background taken into account. For example, to apply such techniques to the large language model, it may be necessary to easily understandably write imperative sentences for labelling and text processing to make a task request. It may also be necessary to prepare information that allows the large language model to correctly understand the context contained in the text data.

### BRIEF DESCRIPTION OF THE DRAWINGS

FIG. 1 is a schematic block diagram illustrating an exemplary configuration of a medical examination support system according to an embodiment;

FIG. 2 is a schematic block diagram illustrating an exemplary functional configuration of an operation terminal according to an embodiment;

FIG. 3 is a schematic block diagram illustrating an exemplary functional configuration of a medical examination support apparatus according to an embodiment;

FIG. 4 schematically illustrates an example of brief-history information according to an embodiment;

FIG. 5 illustrates a table containing exemplary examination-phase information according to an embodiment;

FIG. 6 illustrates an example of imperative-sentence information according to an embodiment;

FIG. 7 schematically illustrates an example of tag candidates according to an embodiment;

FIG. 8 schematically illustrates tag candidate information according to an embodiment;

FIG. 9 is a flowchart illustrating an outline of processing by a medical examination support apparatus according to an embodiment, as an example;

FIG. 10 is a schematic block diagram illustrating an exemplary functional configuration of an operation terminal according to a first modification;

FIG. 11 is a schematic block diagram illustrating an exemplary functional configuration of a medical examination support apparatus according to the first modification; and

FIG. 12 is a flowchart illustrating an outline of processing by the medical examination support apparatus according to the first modification, as an example.

### DETAILED DESCRIPTION

According to an embodiment, a medical examination support apparatus includes processing circuitry. The processing circuitry obtains text information representing different kinds of medical records at different time points, the medical records associated with examination information of a patient. The processing circuitry sets imperative-sentence information (prompt) representing an imperative sentence to be input to a large language model, based on the text information. The processing circuitry determines tag candidate information representing a tag candidate to be added to the text information, based on the imperative-sentence information. The processing circuitry outputs the tag candidate information.

Hereinafter, exemplary embodiments of a medical examination support system will be described in detail with reference to the accompanying drawings. Throughout this disclosure, parts or elements given the same reference numerals are considered to perform similar operations, therefore, redundant descriptions thereof will be omitted when appropriate.

### Embodiment

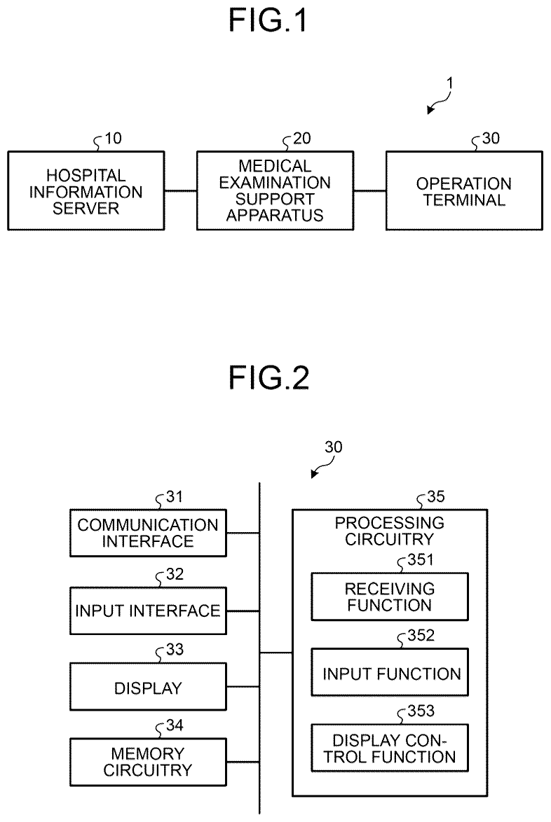

FIG. 1 is a schematic block diagram illustrating an exemplary configuration of a medical examination support system 1 according to an embodiment. The medical examination support system 1 includes a hospital information server 10, a medical examination support apparatus 20, and an operation terminal 30.

The system and the apparatuses are communicably connected to one another via a network. The configuration shown in FIG. 1 is merely exemplary, and the numbers of systems and apparatuses may be optionally changeable. In addition, apparatuses or devices not illustrated in FIG. 1 may be electrically connected to the network.

The hospital information server 10 manages various kinds of information as to patients and electronic medical records for individual patients. The hospital information server 10 represents, for example, a hospital information system (HIS). The hospital information server 10 can be implemented by a computer such as a workstation. The hospital information server 10 stores the electronic medical records in its own memory circuitry. The electronic medical record refers to data containing patient information or patient examination information, for instance.

Examples of patient information of a patient include information contained in the electronic medical record, such as patient ID, gender, age, and clinical history, doctor's finding and observation, and medication history, previously captured medical images of the patient, orders (including examination purpose, an imaging region, an imaging condition, and else) for obtaining medical images, and results of examinations of the patient.

The patient examination information is stored in the form of structured or non-structured data in the electronic medical record. The structured data refers to data in a defined input format, including optionally selectable data such as disease names, symptoms or conditions, drug names, blood test results, and vital data. The non-structured data refers to data in a non-defined input format, such as sentences (natural language text) written in a free comments field or a remarks section of a medical record.

The medical examination support apparatus 20 is an apparatus that provides tag candidates for use in annotation tasks in accordance with a patient's manner of thinking or an examination phase of the patient, with respect to the patient examination information contained in the electronic medical record, which will be described later in detail.

The operation terminal 30 is a terminal to be operated by users including medical professionals as physicians. The operation terminal 30 is an example of a medical processing apparatus. The operation terminal 30 can be implemented by a computer such as a personal computer or a tablet terminal. The operation terminal 30 allows the user to add annotation to the patient examination information (medical record) included in the electronic medical record, for example.

Now, the structure and configuration of the operation terminal 30 according to the present embodiment are explained. FIG. 2 is a schematic block diagram illustrating an exemplary functional configuration of the operation terminal 30 according to the present embodiment. The operation terminal 30 includes, for example, a communication interface 31, an input interface 32, a display 33, memory circuitry 34, and processing circuitry 35. The elements of the operation terminal 30 are not limited to this example.

The communication interface 31 is electrically connected to the processing circuitry 35, to control various data transmissions and communications between the processing circuitry 35 and the apparatuses connected via the network. The communication interface 31 can be, for example, implemented by a network card, a network adaptor, or a network interface controller (NIC).

The input interface 32 is electrically connected to the processing circuitry 35 to receive inputs from the user as an operator (medical professional) to convert the inputs to electrical signals for output to the processing circuitry 35. Thus, the input interface 32 receives and converts an input from the operator into an electrical signal for output to the processing circuitry 35.

The input interface 32 can be, for example, implemented by a trackball, a switch button, a mouse, a keyboard, a touchpad that allows input by touch on the operation surface, a touch screen as an integration of a display screen and a touchpad, non-contact input circuitry including an optical sensor, and audio input circuitry.

In this disclosure the input interface 32 is not limited to the one including physical operational component or components as a mouse and a keyboard. Other examples of the input interface 32 include electrical-signal processing circuitry that receives an electrical signal corresponding to an input from an external input device separated from the operation terminal 30 to output the electrical signal to control circuitry.

The display 33 is electrically connected to the processing circuitry 35 to display various kinds of information and image data output from the processing circuitry 35. The display 33 can be, for example, implemented by a liquid crystal display (LCD), a cathode ray tube (CRT) display, an organic electroluminescence display (OELD), a plasma display, and a touch screen.

The memory circuitry 34 is electrically connected to the processing circuitry 35 and stores various kinds of data therein. The memory circuitry 34 is an example of a storage unit. The memory circuitry 34 stores the patient information. The memory circuitry 34 further stores the examination information associated with the patient information. The memory circuitry 34 further stores tag candidate information as described later. The memory circuitry 34 stores various kinds of computer programs that cause the processing circuitry 35 to retrieve and execute the programs to implement the respective functions. The memory circuitry 34 can be, for example, implemented by a semiconductor memory device as random access memory (RAM) or flash memory, a hard disk, or an optical disk.

The processing circuitry 35 controls the operation of the operation terminal 30 as a whole. The processing circuitry 35 includes, for example, a receiving function 351, an input function 352, and a display control function 353. In some embodiments, the processing and functions of the receiving function 351, the input function 352, and the display control function 353 are individually stored in computer-executable program format in the memory circuitry 34.

Thus, the processing circuitry 35 corresponds to a processor that implements the functions corresponding to the programs by retrieving and executing the programs from the memory circuitry 34. In other words, having retrieved the respective programs, the processing circuitry 35 includes the respective functions shown within the processing circuitry 35 of FIG. 2.

FIG. 2 shows an example that the receiving function 351, the input function 352, and the display control function 353 are implemented by a single processor. Alternatively, the processing circuitry 35 can be constituted of a combination of multiple independent processors, so that the processors can individually execute the programs to implement the respective functions.

Further, in FIG. 2 a single piece of memory circuitry such as the memory circuitry 34 stores computer programs corresponding to the processing and functions, as an example. Alternatively, multiple memory circuits may be disposed in a distributed manner to allow the processing circuitry 35 to retrieve the corresponding programs from the individual memory circuits.

The term “processor” used above signifies, for example, circuitry such as a central processing unit (CPU), a graphical processing unit (GPU), an application specific integrated circuit (ASIC), or a programmable logic device (e.g., a simple programmable logic device (SPLD)), a complex programmable logic device (CPLD), and a field programmable gate array (FPGA).

The processor retrieves and executes the computer programs from the memory circuitry 34 to implement the functions. In place of being stored in the memory circuitry 34, the computer programs may be directly embedded in the circuitry of the processor. In such a case the processor retrieves and executes the computer programs from the circuitry to implement the functions.

The receiving function 351 receives tag candidate information from the medical examination support apparatus 20. The receiving function 351 is one example of a receiver unit. The receiving function 351 also obtains electronic medical records from the hospital information server 10. The tag candidate information from the medical examination support apparatus 20 will be explained later in detail.

The input function 352 inputs annotation-related information with respect to the patient information contained in the electronic medical record. The input function 352 is an example of an input unit. For example, the input function 352 inputs tag information related to an annotation task with respect to the patient information contained in the electronic medical record.

The display control function 353 causes the display 33 to display various kinds of screens. The display control function 353 is an example of a display control unit. The display control function 353 displays, for example, the tag information or the electronic medical record received by the receiving function 351. The display control function 353 displays, for example, the tag information input by the input function 352. In addition to the tag information and the electronic medical record, the display control function 353 may display other information or data.

Conventionally, to input annotation-related tag information to the patient information contained in the electronic medical record, the user performs tagging, using a knowledge base containing an accumulation of medical terms, annotated data, or a rule-based or machine-learning NLP technique, for example. Such techniques tend to basically add the same tags to the same sentences or words so that there may be difficulty in adding tags, taking each patient's background into account. In view of this, the medical examination support apparatus 20 of the present embodiment can provide tag candidates for annotation in accordance with a patient's manner of thinking or the examination phase of the patient.

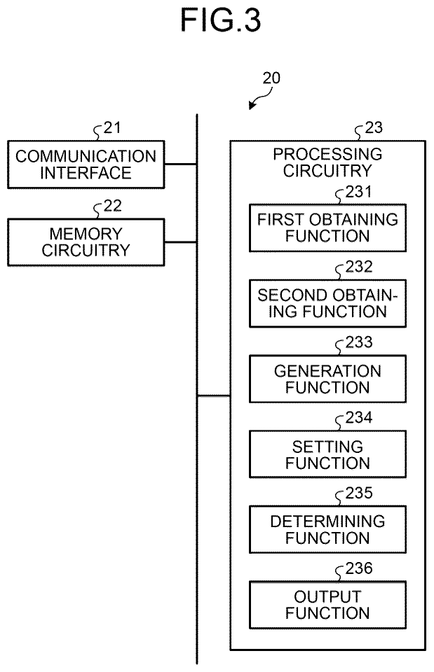

The functional configuration of the medical examination support apparatus 20 according to the present embodiment is now described. FIG. 3 is a schematic block diagram illustrating an exemplary functional configuration of the medical examination support apparatus 20 according to the present embodiment. The medical examination support apparatus 20 includes a communication interface 21, memory circuitry 22, and processing circuitry 23. The functional configuration of the medical examination support apparatus 20 is not limited to this example.

The communication interface 21 is electrically connected to the processing circuitry 23 to control various data transmissions and communications between the processing circuitry 23 and the apparatuses connected via the network. The communication interface 21 can be, for example, implemented by a network card, a network adaptor, or a network interface controller (NIC).

The memory circuitry 22 is electrically connected to the processing circuitry 23 and stores various kinds of data therein. The memory circuitry 22 is an example of a storage unit. The memory circuitry 22 stores patient information. The memory circuitry 22 further stores examination information associated with the patient information. The memory circuitry 22 further stores brief-history information, imperative-sentence information, tag candidates, and tag candidate information, as described later. The memory circuitry 22 stores various kinds of computer programs that cause the processing circuitry 23 to retrieve and execute the programs to implement the respective functions. The memory circuitry 22 can be, for example, implemented by a semiconductor memory device as random access memory (RAM) or flash memory, a hard disk, or an optical disk.

The processing circuitry 23 controls the operation of the medical examination support apparatus 20 as a whole. The processing circuitry 23 includes, for example, a first obtaining function 231, a second obtaining function 232, a generation function 233, a setting function 234, a determining function 235, and an output function 236. In one embodiment the processing and functions of the first obtaining function 231, the second obtaining function 232, the generation function 233, the setting function 234, the determining function 235, and the output function 236 are individually stored in computer-executable program format in the memory circuitry 22.

Thus, the processing circuitry 23 corresponds to a processor that implements the functions corresponding to the programs by retrieving and executing the programs from the memory circuitry 22. In other words, having retrieved the respective programs, the processing circuitry 23 includes the respective functions shown within the processing circuitry 23 of FIG. 3.

FIG. 3 shows an example that the first obtaining function 231, the second obtaining function 232, the generation function 233, the setting function 234, the determining function 235, and the output function 236 are implemented by a single processor. Alternatively, the processing circuitry 23 can be constituted of a combination of multiple independent processors, so that the processors can individually execute the programs to implement the respective functions.

Further, in FIG. 3 a single piece of memory circuitry such as the memory circuitry 22 stores the computer programs corresponding to the processing and functions. Alternatively, multiple memory circuits may be disposed in a distributed manner to allow the processing circuitry 23 to retrieve the corresponding programs from the individual memory circuits.

The term “processor” used above signifies, for example, circuitry such as a central processing unit (CPU), a graphical processing unit (GPU), an application specific integrated circuit (ASIC), or a programmable logic device (e.g., a simple programmable logic device (SPLD)), a complex programmable logic device (CPLD), and a field programmable gate array (FPGA).

The processor retrieves and executes the computer programs from the memory circuitry 22 to implement the functions. In place of being stored in the memory circuitry 22, the computer programs may be directly embedded in the circuitry of the processor. In such a case the processor retrieves and executes the computer programs from the circuitry to implement the functions.

The first obtaining function 231 obtains the patient information from the hospital information server 10. The first obtaining function 231 is one example of an obtainer unit. Specifically, the first obtaining function 231 obtains the patient information from the hospital information server 10 via the network. The first obtaining function 231 stores the patient information in the memory circuitry 22.

The second obtaining function 232 obtains the examination information from the hospital information server 10. The second obtaining function 232 is one example of an obtainer unit. Specifically, the second obtaining function 232 obtains the examination information associated with the patient information from the hospital information server 10 via the network, based on the patient information obtained by the first obtaining function 231. For example, the second obtaining function 232 obtains, as the examination information as to the patient, text information representing different kinds of medical records at different time points, the medical records associated with the patient examination information. The second obtaining function 232 stores the examination information in the memory circuitry 22.

The generation function 233 performs natural language processing to the text information to generate brief-history information based on the text information. The generation function 233 is one example of a generator unit. Specifically, the generation function 233 generates brief-history information based on the text information by performing natural language processing to the text information of different kinds of medical records at different time points, as included in the examination information obtained by the second obtaining function 232. The brief-history information includes, for example, clinical history information representing a patient's clinical history, attitude information representing a patient's manner of thinking, and examination-phase information representing the examination phase of a patient.

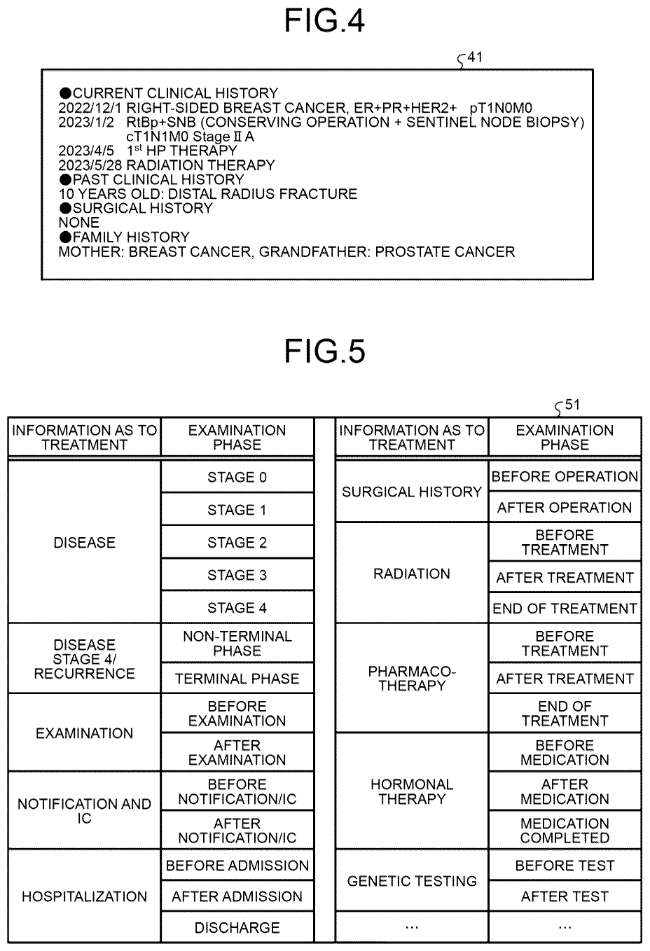

The brief-history information generated by the generation function 233 is now explained with reference to FIG. 4. FIG. 4 schematically illustrates an example of the brief-history information according to an embodiment. FIG. 4 depicts brief-history information 41. The brief-history information 41 is exemplified by clinical history information representing a patient's clinical history in FIG. 4.

In FIG. 4 the brief-history information 41 as clinical history information representing a patient's clinical history includes, for example, a current clinical history, a past clinical history, a surgical history, and a family history. Each of the clinical histories includes information such as date and time, disease names, methods of treatment, and medication information about drug administration. In other words, the clinical history information corresponds to medical and biological brief-history information.

The attitude information as to a patient's manner of thinking contained in the brief-history information 41 describes information related to the patient's external appearance, for example. For instance, a patient suffering breast cancer may consider which of breast conservation therapy and total mastectomy is to undergo. For another example, a patient suffering a tumor may consider whether to undergo a laparotomy resulting in a large wound or an endoscopic operation resulting in a relatively small wound as a surgical procedure to remove the tumor. The attitude information is, for example, information related to an external change of the patient.

Next, the examination-phase information contained in the brief-history information 41 is explained with reference to FIG. 5. FIG. 5 illustrates a table showing an example of the examination-phase information according to an embodiment. The examination-phase information 51 includes, for example, information related to a treatment and an examination phase of the treatment. Examples of the information related to a treatment include disease/illness, recurrence, examination, notification/informed consent (IC), hospitalization, surgical history, radiation, pharmacotherapy, hormonal therapy, and genetic testing.

Referring back to FIG. 3, the setting function 234 sets imperative-sentence information based on the text information. The setting function 234 is one example of a setting unit. Specifically, the setting function 234 sets imperative-sentence information representing imperative sentences to be input to a large language model, based on the text information of different kinds of medical records at different time points, as included in the examination information generated by the second obtaining function 232. For example, the setting function 234 sets the imperative-sentence information representing imperative sentences to be input to a large language model, based on the brief-history information 41 generated by the generation function 233.

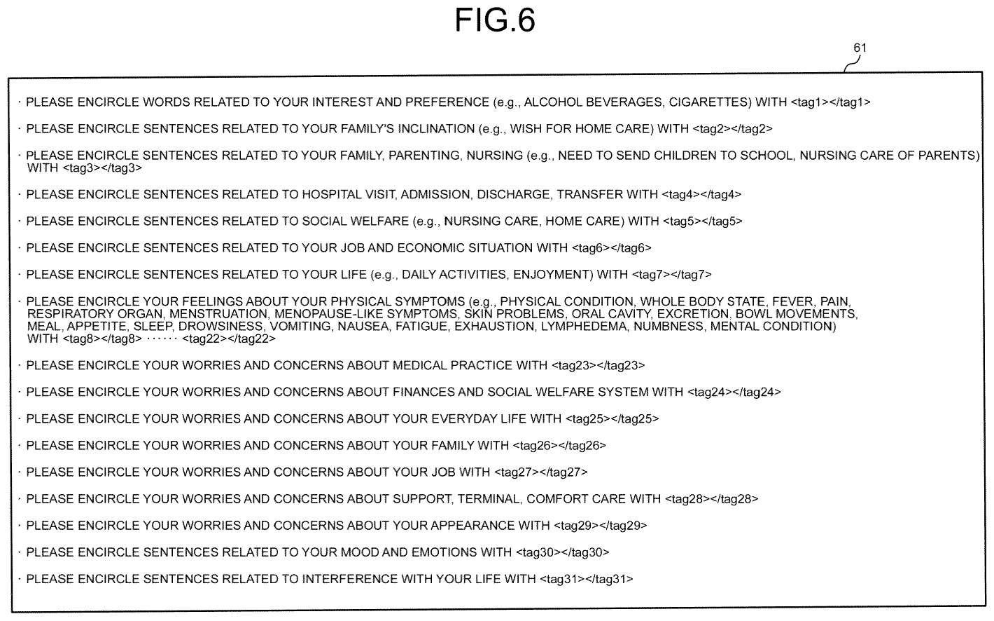

The imperative-sentence information to be set by the setting function 234 is now described referring to FIG. 6. FIG. 6 schematically illustrates the imperative-sentence information according to an embodiment as an example. FIG. 6 depicts imperative-sentence information 61. In FIG. 6 the imperative-sentence information 61 describes imperative sentences based on attitude information as to a patient's manner of thinking.

The imperative-sentence information 61 in FIG. 6 includes attitude information on a patient's manner of thinking. The imperative-sentence information 61 includes, as imperative sentences to be input to a known large language model, tags related to the attitude information representing a patient's manner of thinking. Referring to the imperative-sentence information 61 of FIG. 6, for example, the setting function 234 sets an imperative sentence “Please encircle words related to your interest and preference (attitude information) with <tag1></tag1>”. Thus, the imperative-sentence information 61 includes such tags related to the brief-history information 41. As an example, the imperative-sentence information 61 is information containing tags related to the clinical history information, the attitude information, and the examination-phase information 51.

Returning to FIG. 3, the determining function 235 determines, based on the imperative-sentence information 61, tag candidate information representing tag candidates to be added to the text information. The determining function 235 is one example of a determiner unit. Specifically, the determining function 235 determines tag candidate information representing tag candidates to be added to the text information as to different kinds of medical records at different time points associated with the patient examination information, based on the imperative-sentence information 61 set by the setting function 234.

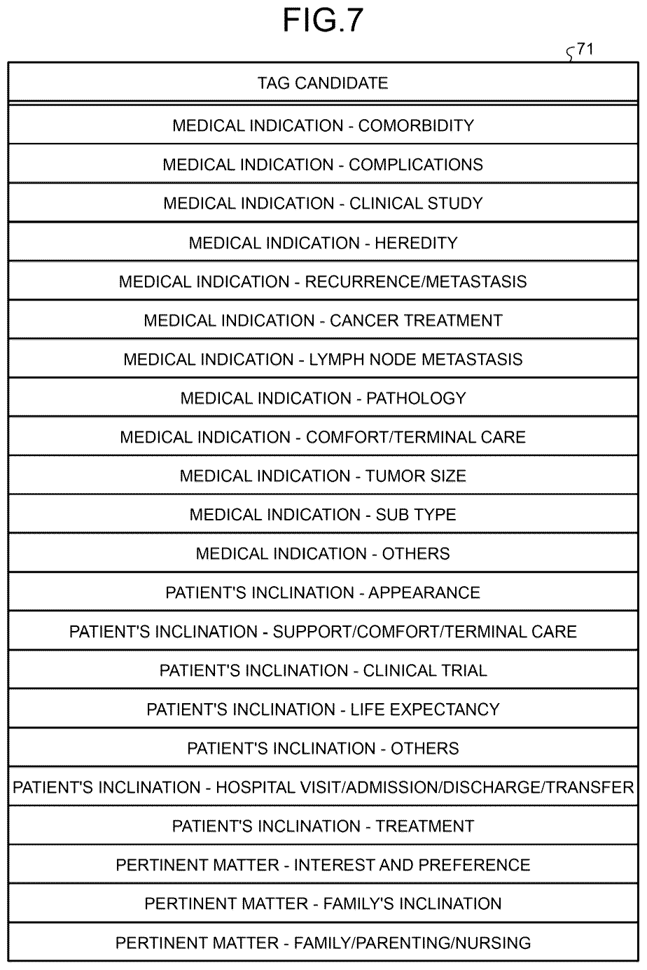

The tag candidates are now described with reference to FIG. 7. FIG. 7 schematically illustrates the tag candidates according to an embodiment as an example. FIG. 7 shows tag candidates 71 for an annotation task. The tag candidates 71 are associated with clinical history information as to a patient's clinical history, attitude information as to a patient's manner of thinking, and examination-phase information 51 as to the examination phase of a patient. The tag candidates may include tags used in the annotation task, in addition to the above examples.

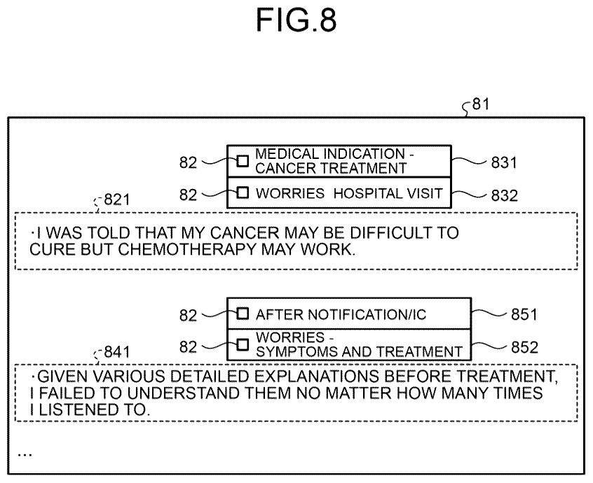

Next, the tag candidate information is explained with reference to FIG. 8. FIG. 8 schematically illustrates tag candidate information according to an embodiment as an example. FIG. 8 shows tag candidate information 81 determined by the determining function 235. The tag candidate information 81 includes text information of different kinds of medical records, tag candidates, and check boxes 82 for allowing a user selection. As illustrated in FIG. 8, for example, the determining function 235 determines a tag candidate 831 and a tag candidate 832 for text information 821 of a medical record. The determining function 235 determines a tag candidate 851 and a tag candidate 852 for text information 841 of the medical record. The tag candidate 831 is linked with the clinical history information as to the patient's clinical history. The tag candidate 832 and the tag candidate 852 are linked with the attitude information as to the patient's manner of thinking. The tag candidate 851 is linked with the examination-phase information 51 as to the examination phase of the patient. The determining function 235 determines the tag candidate information 81 representing tag candidates to be added to the text information contained in the different kinds of medical records at different time points.

Returning to FIG. 3, the output function 236 outputs the tag candidate information 81. The output function 236 is one example of an output unit. Specifically, the output function 236 outputs the tag candidate information 81 determined by the determining function 235. Thereby, the medical examination support apparatus 20 of the present embodiment can provide the tag candidates for use in the annotation task in dependence on the patient's manner of thinking or the examination phase of the patient.

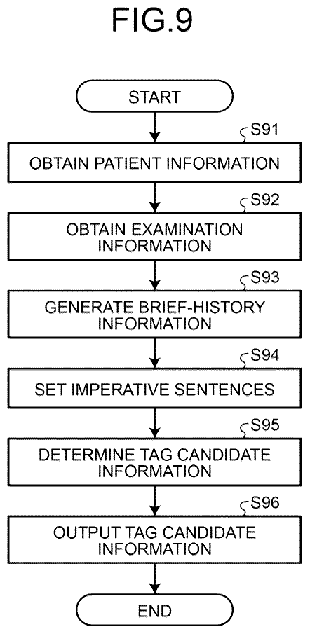

Next, an exemplary process to be performed by the medical examination support apparatus 20 according to an embodiment is explained. FIG. 9 is a flowchart illustrating outlined processing to be performed by the medical examination support apparatus 20 according to an embodiment, as an example. For the sake of specificity, the hospital information server 10 is defined to store electronic medical records for use in annotation tasks herein.

First, the first obtaining function 231 obtains patient information from the hospital information server 10 (step S91). The second obtaining function 232 then obtains examination information associated with the patient information from the hospital information server 10, based on the patient information obtained by the first obtaining function 231 (step S92). The generation function 233 then performs natural language processing to the text information of different kinds of medical records at different time points, as contained in the examination information obtained by the second obtaining function 232, to generate brief-history information 41 based on the text information (step S93).

The setting function 234 sets imperative-sentence information 61 representing imperative sentences to be input to a large language model, on the basis of the brief-history information 41 generated by the generation function 233 (step S94). The determining function 235 then determines tag candidate information 81 representing tag candidates to be added to the text information of the different kinds of medical records at different time points, on the basis of the imperative-sentence information 61 set by the setting function 234 (step S95). The output function 236 then outputs the tag candidate information 81 determined by the determining function 235 (step S96). This completes the processing circuitry 23's processing.

As such, the medical examination support apparatus 20 of an embodiment obtains the text information representing different kinds of medical records at different time points associated with the patient examination information, and performs natural language processing to the text information to generate the brief-history information 41 based on the text information. The medical examination support apparatus 20 then sets, based on the brief-history information 41, the imperative-sentence information 61 including imperative sentences to be input to the large language model. The medical examination support apparatus 20 determines, for output, the tag candidate information 81 representing tag candidates to be added to the text information, based on the imperative-sentence information 61.

According to at least one of the embodiments described above, the medical examination support apparatus 20 can provide the tag candidates for use in the annotation task for a medical record in accordance with the patient's manner of thinking or the examination phase of the patient by outputting the tag candidate information 81 including tag candidates to be added to the text information representing different kinds of medical records at different time points associated with the patient examination information, for example. This further allows the user to conduct an annotation task while checking the output of the tag candidate information 81, leading to reducing the user's workloads involving the annotation task. In addition, the medical examination support apparatus 20 can output the tag candidate information 81 suited for each patient, enabling the user to perform annotation tasks specific to individual patients.

By performing the annotation task specific to each patient, the user can utilize the examination information used in the annotation task to create a patient journey, which represents a process of a series of the patient's overall actions including recognition of a disease or symptoms, a visit to a medical facility, and receipt of drug administration and/or medical treatment.

The above-described embodiments can be modified or changed when appropriate by partially changing the elements or functions of the respective apparatuses. Thus, some modifications of the above embodiments will be explained as other embodiments in the following. The following will mainly describe differences from the above-described embodiments, and a description of the common features given the same reference numerals will be omitted. In addition, the other embodiments as explained below may be implemented individually or in combination when appropriate.

### First Modification

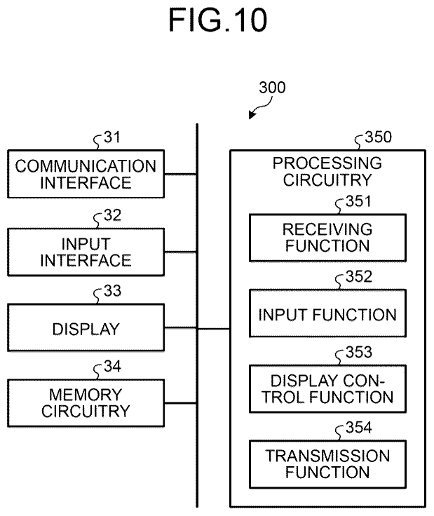

According to one of the above embodiments, the processing circuitry in the medical examination support apparatus may obtain user feedback about the tag candidate information 81 output from the output function 236. FIG. 10 is a schematic block diagram illustrating an exemplary functional configuration of an operation terminal 300 according to a first modification. Processing circuitry 350 of the operation terminal 300 of the first modification includes, for example, a receiving function 351, an input function 352, a display control function 353, and a transmission function 354. In the first modification, the processing and functions of the receiving function 351, the input function 352, the display control function 353, and the transmission function 354 are stored in computer-executable program format in the memory circuitry 34.

The transmission function 354 transmits, to the medical examination support apparatus, feedback information representing feedback about the tag candidate information 81. The transmission function 354 is one example of a transmitter unit. Specifically, in response to user feedback relative to the tag candidate information 81 output from the medical examination support apparatus, the transmission function 354 transmits feedback information indicative of the user feedback about the tag candidate information 81.

For example, referring to the tag candidate information 81 output from the medical examination support apparatus, the user inputs tag information for use in an annotation task with respect to patient information contained in an electronic medical record. During the annotation task, the user may desire to correct the tag candidate information and output the corrected tag candidate information from the medical examination support apparatus. In view of this, in the first modification the transmission function 354 of the processing circuitry 350 included in the operation terminal 300 transmits such feedback information representing user feedback about the tag candidate information to the medical examination support apparatus.

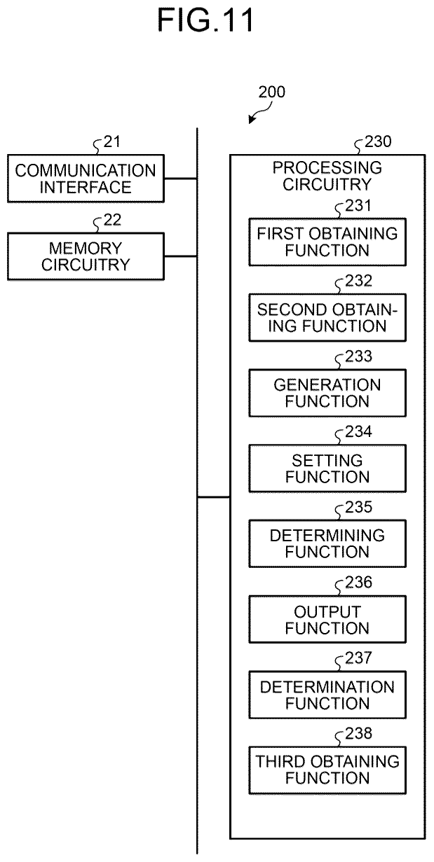

FIG. 11 is a schematic block diagram illustrating an exemplary configuration of a medical examination support apparatus 200 according to the first modification. Processing circuitry 230 in the medical examination support apparatus 200 of the first modification includes, for example, a first obtaining function 231, a second obtaining function 232, a generation function 233, a setting function 234, a determining function 235, an output function 236, a determination function 237, and a third obtaining function 238. The processing and functions of the first obtaining function 231, the second obtaining function 232, the generation function 233, the setting function 234, the determining function 235, the output function 236, the determination function 237, and the third obtaining function 238 are stored in computer-executable program format in the memory circuitry 22.

The determination function 237 determines whether feedback information is available. The determination function 237 is one example of a determination unit. Specifically, the determination function 237 determines whether any feedback information has been transmitted from the operation terminal 300.

The third obtaining function 238 obtains the feedback information, if any. The third obtaining function 238 is one example of an obtainer unit. Specifically, the third obtaining function 238 obtains the feedback information as transmitted from the operation terminal 300. The third obtaining function 238 then stores the feedback information in the memory circuitry 22.

The setting function 234 sets again the imperative-sentence information containing imperative sentences to be input to the large language model, on the basis of the brief-history information generated by the generation function 233 and the feedback information obtained by the third obtaining function 238.

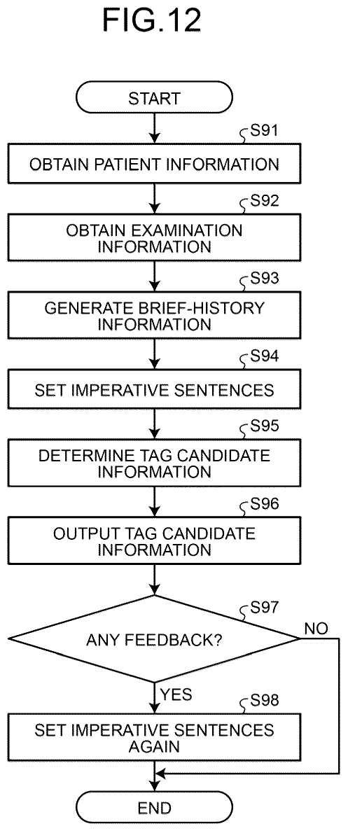

Next, exemplary processing to be performed by the medical examination support apparatus 200 according to the first modification is explained. FIG. 12 is a flowchart illustrating an outline of processing to be performed by the medical examination support apparatus 200 according to the first modification, as an example. Note that a description of the processing common to that of the medical examination support apparatus 20 of an embodiment in FIG. 9 is omitted.

At step S97, the determination function 237 determines whether there is any feedback information transmitted from the operation terminal 300 (step S97). When the determination function 237 determines that no feedback information has been transmitted from the operation terminal 300 (No at step S97), the processing circuitry 23 ends the processing. When the determination function 237 determines that the feedback information has been transmitted from the operation terminal 300 (Yes at step S97), the processing proceeds to step S98.

At step S98, the setting function 234 sets again the imperative-sentence information containing imperative sentences to be input to the large language model, on the basis of the brief-history information generated by the generation function 233 and the feedback information obtained by the third obtaining function 238 (step S98).

The medical examination support apparatus 200 can perform the series of steps as above while the user including the operator (a medical professional, as an example) is writing a medical record. Further, the medical examination support apparatus 200 can display tag candidates on the display at the time of storing the medical record in the hospital information server 10, allowing the user to place a check mark in one or more of the check boxes 82 for storage.

As described above, in response to receipt of the feedback information about the generated tag candidate information 81, the medical examination support apparatus 200 of the first modification sets again the imperative-sentence information containing imperative sentences to be input to the large language model, based on the generated brief-history information and the obtained feedback information. As a result, by providing feedback about the output of the tag candidate information 81, the user can allow the medical examination support apparatus to output the tag candidate information more fitted to each patient. Thereby, the user can perform annotation tasks more adapted to individual patients.

### Second Modification

The above embodiments have described, but are not limited to, an example that the tag candidates include the clinical history information representing a patient's clinical history, the attitude information representing a patient's manner of thinking, and the examination-phase information representing the examination phase of a patient. For example, the tag candidates may include future-potential tag candidates.

The future-potential tag candidate refers to a tag candidate signifying that a patient's adverse prognosis (e.g., 10% survival rate) is inferred from his or her current clinical history and a change of the condition may be forthcoming. A change of the condition refers to, for example, a change of a current state to a state as recovery or exacerbation, or maintenance of a current state. In addition, the tag candidates may include attitude information of a patient's family representing a manner of their thinking or their inclinations.

### Third Modification

The above embodiments have described, but are not limited to, an example that the medical examination support apparatus 20 obtains the text information representing different kinds of medical records at different time points associated with the patient examination information to perform natural language processing to the text information and generate the brief-history information 41 based on the text information, and sets the imperative-sentence information 61 representing imperative sentences to be input to a large language model, based on the brief-history information 41. The medical examination support apparatus 20 of a third modification may obtain text information of different kinds of medical records at different time points associated with patient examination information and set imperative-sentence information 61 representing imperative sentences to be input to a large language model, based on the text information.

For example, the setting function 234 of the medical examination support apparatus 20 sets imperative-sentence information representing imperative sentences to be input to a large language model, based on the text information of different kinds of medical records at different time points as included in the examination information obtained by the second obtaining function 232. The operation and effects of the third modification are similar to or the same as those in some embodiments, therefore, a description thereof is omitted.

Note that the elements of the respective apparatuses of some embodiments shown in the drawings are functional and conceptual, and they may not be physically configured as depicted in the drawings. Thus, all or part of the individual apparatuses can be configured in a distributed or integrated manner functionally or physically in any units depending on various kinds of load or a status of use, in addition to the manner of distribution/integration shown in the drawings. In addition, the processing and functions of the respective apparatuses can be partially or entirely implemented by a CPU and computer programs to be analyzed and executed by the CPU, or implemented as hardware by wired logic.

To implement the technical ideas of one embodiment by a medical examination support method, the medical examination support method may include obtaining text information representing different kinds of medical records at different time points, the medical records associated with examination information of a patient; performing natural language processing to the text information to generate brief-history information based on the text information; setting imperative-sentence information representing imperative sentences to be input to a large language model, based on the brief-history information; determining tag candidate information representing tag candidates to be added to the text information, based on the imperative-sentence information; and outputting the tag candidate information. The procedure and effects of the medical examination support method are similar to or the same as those in some embodiments, therefore, a description thereof is omitted.

The methods described herein can be implemented by execution of prepared computer programs using a computer such as a personal computer or a workstation. Such programs can be distributed via a network as the Internet. Further, these programs can be also recorded on a non-transitory computer-readable recording medium, such as a hard disk, a flexible disk, (FD), a CD-ROM, an MO disk, or a DVD, to be retrieved and executed by a computer from the recording medium.

According to at least one of the embodiments as above, it is made possible to provide tag candidates to be used in annotation tasks for medical records in accordance with each patient's manner of thinking or the examination phase of the patient.

While certain embodiments have been described, these embodiments have been presented by manner of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical examination support apparatus comprising processing circuitry configured to:
obtain text information representing different kinds of medical records at different time points, the medical records associated with examination information of a patient,
set imperative-sentence information representing an imperative sentence to be input to a large language model, based on the text information,
determine tag candidate information representing tag candidate to be added to the text information, based on the imperative-sentence information, and
output the tag candidate information.

2. The medical examination support apparatus according to claim 1, wherein
the processing circuitry is configured to obtain patient information of the patient, and obtain the examination information associated with the patient information, based on the patient information.

3. The medical examination support apparatus according to claim 1, wherein the processing circuitry is configured to:
perform natural language processing to the text information to generate brief-history information based on the text information, and
set the imperative-sentence information representing the imperative sentence to be input to the large language model, based on the brief-history information.

4. The medical examination support apparatus according to claim 1, wherein
the examination information is in the form of structured data or non-structured data,
the structured data is data in a defined input format, and
the non-structured data is data in a non-defined input format.

5. The medical examination support apparatus according to claim 3, wherein
the brief-history information includes clinical history information representing a clinical history of the patient, attitude information representing a manner of thinking of the patient, and examination-phase information representing an examination phase of the patient.

6. The medical examination support apparatus according to claim 5, wherein
the tag candidate information represents tag candidates associated with the clinical history information, the attitude information, and the examination-phase information.

7. The medical examination support apparatus according to claim 5, wherein
the clinical history information includes at least one of a current clinical history, a past clinical history, a clinical history related to a surgical history and a family history, and
the clinical history includes at least one of a date and time, a disease name, a method of treatment, and medication information about drug administration.

8. The medical examination support apparatus according to claim 5, wherein
the attitude information is information related to an external change of the patient.

9. The medical examination support apparatus according to claim 5, wherein
the examination-phase information includes information related to a treatment, and
the information related to a treatment includes at least one of a disease, recurrence, examination, a notification, hospitalization, a surgical history, radiation, pharmacotherapy, hormonal therapy, and genetic testing.

10. The medical examination support apparatus according to claim 6, wherein
the tag candidate information includes the text information, the tag candidates, and a check box for allowing a user selection.

11. The medical examination support apparatus according to claim 3, wherein the processing circuitry is configured to:
obtain feedback information representing feedback about the tag candidate information, and
set the imperative-sentence information again based on the brief-history information and the feedback information.

12. A medical examination support method comprising:
obtaining text information representing different kinds of medical records at different time points, the medical records associated with examination information of a patient;
setting imperative-sentence information representing an imperative sentence to be input to a large language model, based on the text information;
determining tag candidate information representing tag candidates to be added to the text information, based on the imperative-sentence information; and
outputting the tag candidate information.

13. A non-transitory computer-readable medium storing instructions which, when executed by a computer, cause the computer to perform:
obtaining text information representing different kinds of medical records at different time points, the medical records associated with examination information of a patient;
setting imperative-sentence information representing an imperative sentence to be input to a large language model, based on the text information;
determining tag candidate information representing tag candidates to be added to the text information, based on the imperative-sentence information; and
outputting the tag candidate information.
